(19) United States
(12) Patent Application Publication
(10) Pub. No.: US 2005/0221397 A1
(43) Pub. Date: Oct. 6, 2005

(54) RM2 antigen (beta1,4-GalNAc-disialyl-Lc4) as prostate cancer-associated antigen

(75) Inventor: Seiichi Saito, Aoba-ku (JP)

Correspondence Address: SUGHRUE MION, PLLC, 2100 PENNSYLVANIA AVENUE, N.W., SUITE 800, WASHINGTON, DC, 20037, US

(73) Assignee: NORTHERN ADVANCEMENT CENTER FOR SCIENCE & TECHNOLOGY

(21) Appl. No.: 10/812,357

(22) Filed: Mar. 30, 2004

(51) Int. Cl. G01N 33/53; G01N 33/567; G01N 33/574

(52) U.S. Cl. 435/7.23

(57) ABSTRACT

A novel carbohydrate antigen, β1,4-GalNAc-disialyl-Lc₄, defined by monoclonal antibody RM2, is expressed in human prostate cancer, but not in benign prostate hypertrophy (BPH) or normal prostate gland. Monoclonal antibody RM2 or other antibodies with similar specificity are useful for diagnosis of prostate cancer by immunohistology of biopsy samples, specifications from a total prostatectomy, and quantitative determination of RM2 antigen in sera of patients.

### FIELD OF THE INVENTION

The instant invention relates to the identification of a specific carbohydrate antigen as a human prostate cancer-associated antigen.

### BACKGROUND OF THE INVENTION

Prostate cancer in the United States is diagnosed every 2.75 minutes; over 230,000 new cases occur each year. Prostate cancer is the most commonly diagnosed cancer among men (over 32% of all new cancer cases), and an estimated 29,900 men die from prostate cancer each year. It has the highest incidence, in the U.S., of any type of cancer. Similar trends are observed in other advanced countries.

Prostate-specific antigen (PSA) is used currently for diagnosis of prostate cancer, because an increase in its serum level (>6.1 ng per ml) is often associated with the disease. However, PSA is a protein antigen and is found in normal prostate glands as well as in prostate cancer. Increased PSA level is also associated with benign prostate hypertrophy (BPH) and prostatitis, and is therefore not a conclusive indicator of prostate cancer.

Aberrant glycosylation (formation of abnormal carbohydrate chains at the cell surface) occurs in many types of cancer. The pattern of aberrant glycosylation, and the expression of specific glycosyl epitopes associated with specific types of cancer, have been used as criteria for diagnosis of many types of human cancer.

There has been a search for abnormal carbohydrate chains whose expression is associated with human prostate cancer, but not with normal prostate or BPH. This invention relates to identification of such a structure, termed “RM2 antigen” (β1,4-GalNAc-disialyl-Lc₄), which specifically binds to monoclonal antibody (mAb) RM2.

### SUMMARY OF THE INVENTION

This invention is based on results from a previous search for renal cell carcinoma (RCC) antigen, as applied in studies of prostate cancer. Originally, RCC cell line TOS 1 was used as an immunogen to obtain a mAb that reacts with RCC; this mAb was termed “RM2”. The structure of the antigen recognized by RM2 was later identified as β1,4-GalNAc-disialyl-Lc₄) (FIG. 1). RCC is a relatively rare type of cancer, and not all RCCs express this structure. Since organs of the urogenital system have a common embryonic development, a systematic examination was conducted of RM2 antigen expression in 35 cases of prostate cancer, representing various stages of the disease. All of these 35 cases showed positive reactivity with mAb RM2, i.e., presence of RM2 antigen. 18 cases were moderately or strongly positive; 17 cases were weakly positive. Negative or very weak staining was observed in normal glands of all 35 cases, and no staining was observed in 6 cases of BPH. Median Gleason scores (an indicator of malignancy) were 8 and 7, respectively, for the moderately/strongly positive and weakly positive cases.

Negative RM2 expression in BPH has special relevance for diagnostic application of RM2. In the PSA assay, slightly to moderately elevated values (4-10 ng/ml) are often associated with BPH. Since RM2 is not expressed in BPH, the ability of RM2 to distinguish prostate cancer from BPH will be extremely useful in selecting biopsy cases among men with elevated PSA in the range of 4-10 ng/ml, using a serum RM2 test. Out of 9 radical prostatectomy specimens, 5 showed moderately/strongly positive (m/s) staining, and 4 showed weakly positive (w) staining. 4 of the 5 cases of m/s staining were pathologically non-organ confined, whereas 4 of the 4 cases of w staining were organ-confined. Although the number of cases examined was small, there is clear correlation between RM2 positivity and pathological stage (p<0.02). Prediction of pathological stage in clinically localized prostate cancer is very important in choosing between treatment options, i.e., radical prostatectomy vs. radiation therapy. These data indicate that RM2 may also be useful to predict the pathological stage in clinically localized prostate cancer, in which pathologically non-organ confined cancer is found in about 40% of contemporary radical prostatectomy series.

According to contemporary data, the majority of male patients undergoing PSA testing showed PSA values of 4-10 ng/ml. Yet, only 25% of patients having PSA values in this range were found to have prostate cancer by biopsy, i.e., >70% of patients with a “high” PSA value did not have prostate cancer. Use of this test, worldwide, represents a tremendous waste of money, time, and labor, and psychological stress on patients.

For this reason, discovery of a specific antigen whose expression is associated with human prostate cancer, but not with normal prostate or BPH, is a very important medical advance. The present invention provides a method for diagnosing prostate cancer, comprising detecting the presence of or elevated levels of RM2 antigen, have the epitope structure shown below, in a specimen from a patient suspected of having prostate cancer, 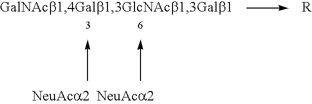
wherein R represents a carrier.

In a preferred embodiment, the RM2 antigen is detected with an antibody that specifically binds to RM2 antigen. In a more preferred embodiment, the antibody is RM2 monoclonal antibody.

The present invention also provides a kit for diagnosing prostate cancer, comprising:
(a) At least one moiety that specifically binds to RM2 antigen, having the epitope structure shown below, from a specimen obtained from a patient suspected of having prostate cancer: 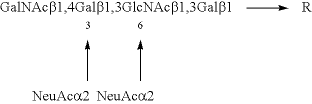
   wherein R represents a carrier,
(b) Instructions for diagnosing prostate cancer using said kit, and
(c) Optionally, a means for detecting the presence of said antigen by specific binding of said moiety to said antigen.

### BRIEF DESCRIPTION OF THE FIGURES

FIG. 1. Structure of RM2 antigen.

FIG. 2. Immunohistological patterns of RM2 antigen expression in biopsy samples of prostate cancer having various Gleason scores. Panel A: sample 1 (Gleason score 5+4). Panel B: sample 2 (Gleason score 4+5). Panel C: sample 3 (Gleason score 5+4). Panel D: sample 4 (Gleason score 3+3). Panel E: sample 5 (Gleason score 4+3). Panel F: sample 6 (Gleason score 4+3). Magnification x200. Negative or only a weak RM2 immunostaining was observed in normal glands.

FIG. 3. Immunohistological patterns of RM2 antigen expression in radical prostatectomy specimens. Panel A: sample 1 (Gleason score 3+4). Panel B: sample 2 (Gleason score 4+4). Panel C: sample 3 (Gleason score 4+5). Magnification x200. ms: moderate/strong, w: weak, RP: radical prostatectomy.

FIG. 4. Immunohistological patterns of RM2 antigen expression in cases of BPH (Panel A) and normal prostate (Panel B) from radical prostatectomy specimens. No RM2 immunostaining was observed in BPH, and only a weak RM2 immunostaining was observed in normal glands. Magnification x100.

FIG. 5. Western blot analysis of prostate cancer cell lines by RM2. Panel A immunostaining by RM2, Panel B: immunostaining by mouse IgM (negative control). 1. PC3 (5 μg), 2. LNCap (5 μg), 3. PC3 (10 μg), 4. LNCap (10 g), 5. PC3 (15 μg) 6. LNCap (15 μg), 7. PC3 (20 μg) 8. LNCap (20 μg), M: size marker. RM2 detected 49 kDa glycoprotein as the major band, in addition to several other bands in LNCap and PC3.

### DETAILED DESCRIPTION OF THE INVENTION

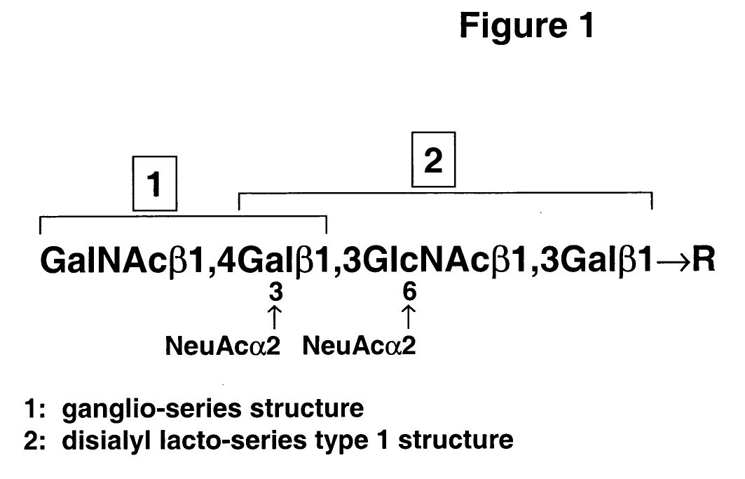

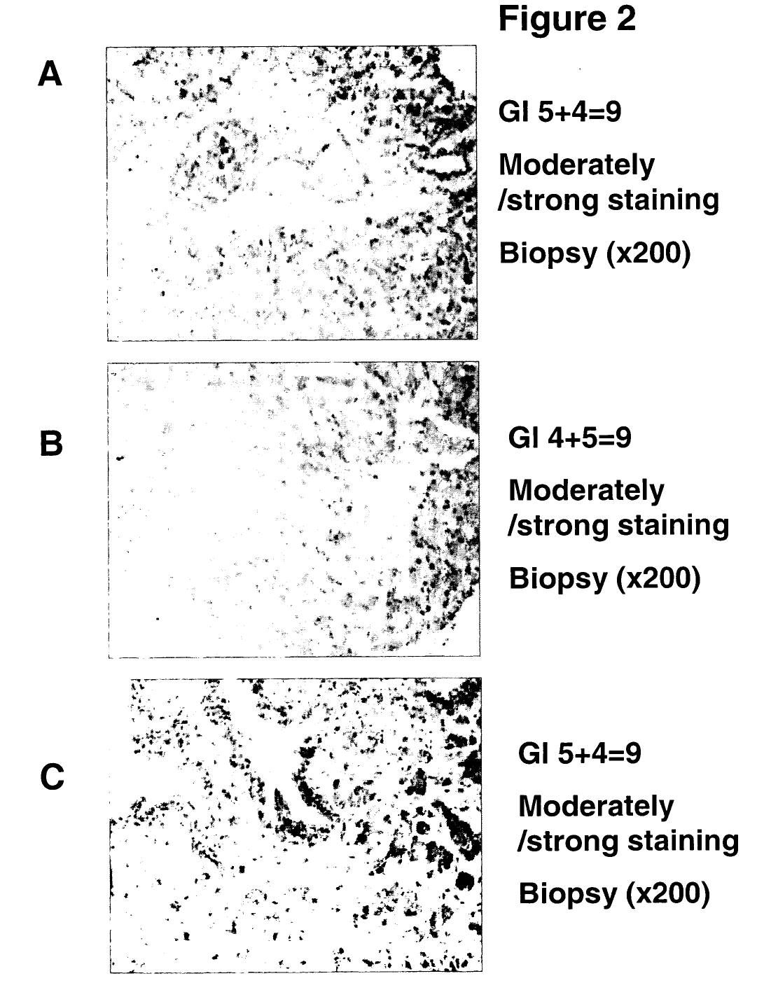

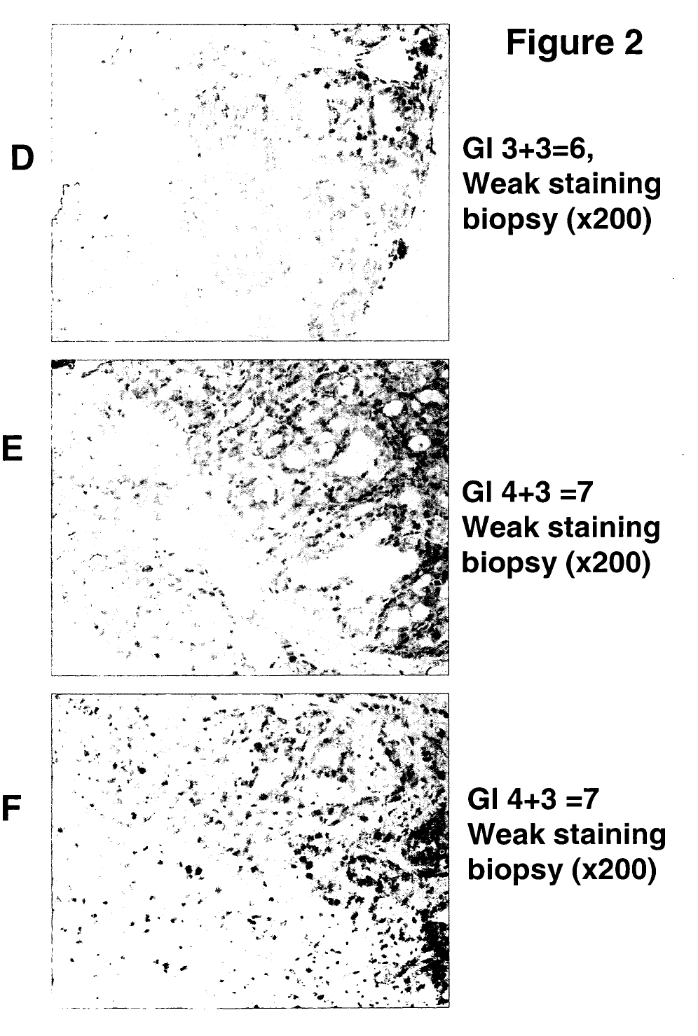

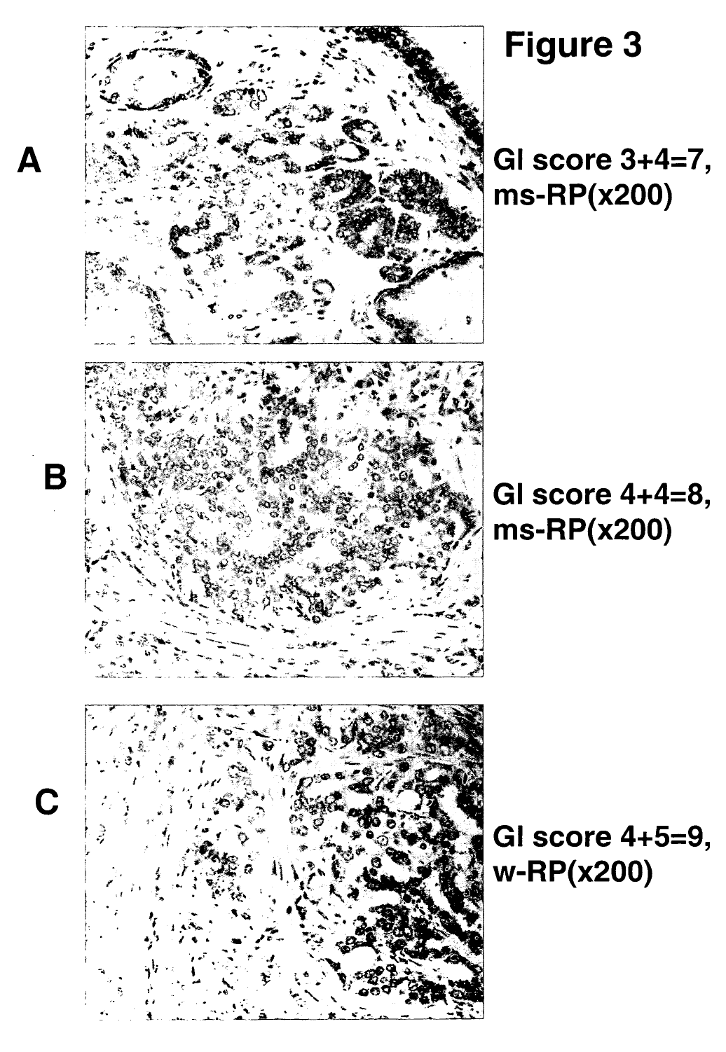

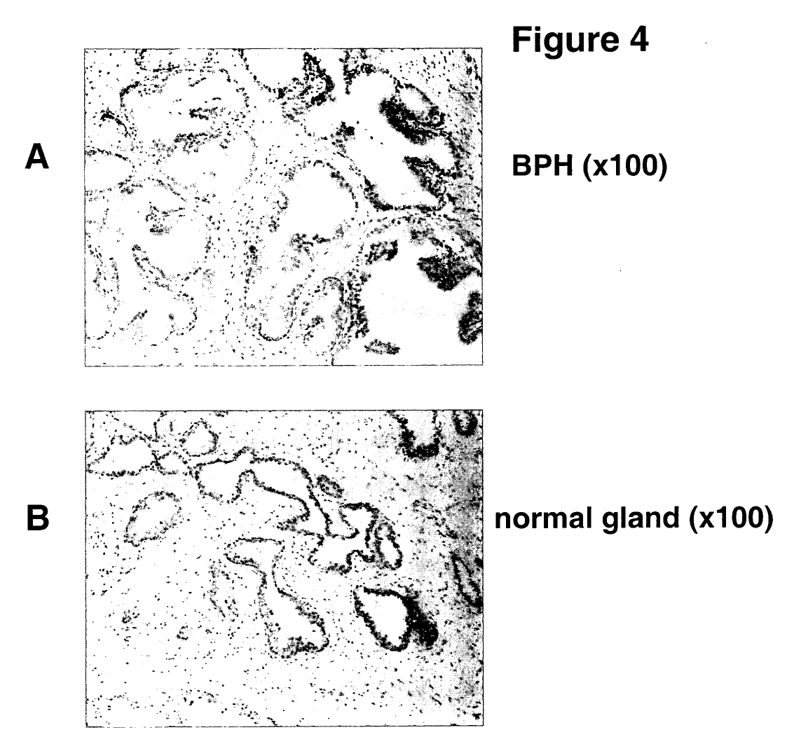

A. RM2 antigen and antibodies. Based on the general concept that human tumors are characterized by expression of specific carbohydrate antigens, bound either to glycosphingolipid or to glycoprotein (Hakomori, S. 1989 Adv. Cancer Res. 52, 257-331; Hakomori, S. 1996 Cancer Res. 56, 5309-5318), the presence of slow-migrating gangliosides highly expressed in RCC was demonstrated (Saito, S., Orikasa, S., Ohyama, C., Satoh, M., and Fukushi, Y. (1991) Int. J. Cancer 49, 329-334). Monoclonal antibody RM2 was established by immunization of mice with RCC cell line TOS 1, followed by repeated cloning of hybridoma secreting antibody that recognized slow-migrating gangliosides expressed in RCC tissue (Saito, S., Levery, S. B., Salyan, M. E. K., Goldberg, R. I., and Hakomori, S. 1994 J. Biol. Chem. 269, 5644-5652). Further systematic studies on the structure of the antigen recognized by mAb RM2, termed “RM2 antigen,” by 1- and 2-dimensional ¹H-NMR and mass spectrometry clarified it as β1,4-GalNAc-disialyl-Lc₄ (FIG. 1). The structure is highly novel and consists of “ganglio-series” (region 1 in FIG. 1) and “disialyl lacto-series type 1 chain” (region 2 in FIG. 1) groups (Ito, A., Levery, S. B., Saito, S., Satoh, M., and Hakomori, S. 2001 J. Biol. Chem. 276, 16695-16703).

B. RM2 antigen as prostate cancer-associated antigen. Since urogenital tissues and organs are ontogenically related, the present inventors hypothesized that antigen expressed in RCC may also be expressed in other urogenital cancers, particularly prostate cancer, which has the highest incidence and mortality. Preliminary studies were conducted on biopsy samples from 40 prostate cancer cases. Biopsy samples included all stages of the cancer, most of which were advanced stages. That is, about 66% of the biopsy samples were obtained from patients with “non-organ confined” prostate cancer. Tissues were formalin-fixed and paraffin-embedded for standard histology procedure. Out of the 40 samples, 35 showed good preservation of structure, and immunohistology results could be evaluated. All of these 35 cases showed positive reactivity with RM2 antibody. 18 cases were moderately or strongly positive; 17 cases were weakly positive. These cases are described below.

Biopsy Specimens

1. 18 Moderately/Strongly Positive Cases

age (median): 72.5 yrs
PSA value (median): 40 ng/ml (range 2.5-3797 ng/ml)
Gleason score (median): 8 (for 18 total cases)
   score 6 in 3 cases
   score 7 in 5 cases
   score 8 in 3 cases
   score 9 in 7 cases
clinical stage
   degree of localization (T)
      T2 and lower than T2: 6 cases
      T3 and higher than T3: 12 cases
   with metastasis
      stage D2 (metastasis to bone or to distant lymph nodes, beyond regional lymph nodes): 5 cases
      stage D1 (metastasis to regional lymph nodes): 1 case
   without metastasis
   T1c˜T4N0M0: 12 cases
   (T1c: 4 cases, T2: 2 cases, T3: 5 cases, T4: 1 case)
      2. 17 Weakly Positive Cases
age (median): 71 yrs
PSA value (median): 37 ng/ml (range 7-1723 ng/ml)
Gleason score (median): 7 (for 17 total cases)
score 6 in 3 cases
   score 7 in 7 cases
   score 8 in 4 cases
   score 9 in 3 cases
clinical stage
   degree of localization (T)
      T2 and lower than T2: 6 cases
      T3 and higher than T3: 11 cases
   with metastasis
      stage D2 (metastasis to bone or to distant lymph nodes, beyond regional lymph nodes): 4 cases
      stage D1 (metastasis to regional lymph nodes): 1 case
   without metastasis
      T1c˜T3N0M0: 12 cases
      (T1c: 3 cases, T2: 3 cases, T3: 6 cases)
         Radical Prostatectomy Specimens (9 Total)
age (median): 65 yrs
PSA (median): 6.1 ng/ml (range 4.4-13.2 ng/ml)
   1. 5 Moderately/Strongly Positive cases
Gleason score 7: 3 cases
Gleason score 8: 1 case
Gleason score 9: 1 case
non-organ confined (pT3 and higher than pT3): 4 cases
organ confined (pT2 and lower than pT2): 1 case
   2. 4 Weakly Positive Cases
Gleason score 8: 2 cases
Gleason score 9: 2 cases
organ confined (pT2 and lower than pT2): 4 cases

C. RM2 antigen as glycoprotein of tumor cells. RM2 antigen was originally found as glycosphingolipid (disialoganglioside), as described in Section A. above. However, some of this antigen present in tumor cells can be detected by sodium dodecyl sulfate polyacrylamide gel electrophoresis (SDS-PAGE) followed by Western blot analysis. Briefly, cells were put on ice, rinsed with ice-cold PBS, and lysed with cell lysis buffer (20 mM Tris PH 7.4, 150 mM NaCl, 2 mM EDTA, 1% NP40, 50 mM NaF, 10 μg/ml aprotinin, 10 μg/ml leupeptin, 1 mM PMSF, 1 mM Na₃VO₄). The extracts were clarified by centrifugation at 12,000 rpm for 5 min. Lysates containing equal amounts of proteins were resolved by electrophoresis on 10% SDS-PAGE and then transferred to Hybond P PVDF membrane (Amersham Biosciences). Membranes were blocked with TBS-Tween containing 1% BSA, then incubated with primary antibodies. Bound antibodies were detected using appropriate peroxidase-coupled secondary antibodies, followed by enhanced chemiluminescent detection system (ECL, Boehringer Mannheim).

An important point is that glycoprotein antigens are released from cells more easily than glycosphingolipid antigens. Many tumor-associated antigens used as diagnostic probes during serum examination are glycoproteins rather than glycosphingolipids.

D. Method for diagnosis of prostate cancer: The present invention provides a method for diagnosing prostate cancer, comprising detecting the presence of or elevated levels of RM2 antigen, having the epitope structure shown below, in a specimen from a patient suspected of having prostate cancer: 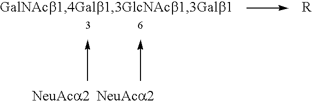
wherein R represents a carrier.

The method is especially useful for distinguishing BPH from malignant prostate cancer.

Suitable carriers include (i) lactosamine chain N-linked or O-linked to glycoprotein, (ii) 4Glcβ1-1Cer in glycosphingolipid, or (iii) any other naturally-occurring or synthetic carrier molecule.

Specimens suitable for use in diagnosing prostate cancer include a biopsy of cancerous prostate tissue, a specimen from a total prostatectomy, and serum.

According to the present invention, the method for diagnosis of prostate cancer can be any method capable of detecting the presence of or elevated levels of RM2 antigen in a specimen in which the presence of or elevated levels of the antigen correlates with the occurrence of prostate cancer. Examples of methods for detecting the presence of or elevated levels of RM2 antigen include “sandwich” immunoassays, electrospray ionization (ESI) and matrix-assisted laser desorption/ionization (MALDI) mass spectrometry (MS), and surface plasmon resonance (SPR) spectroscopy.

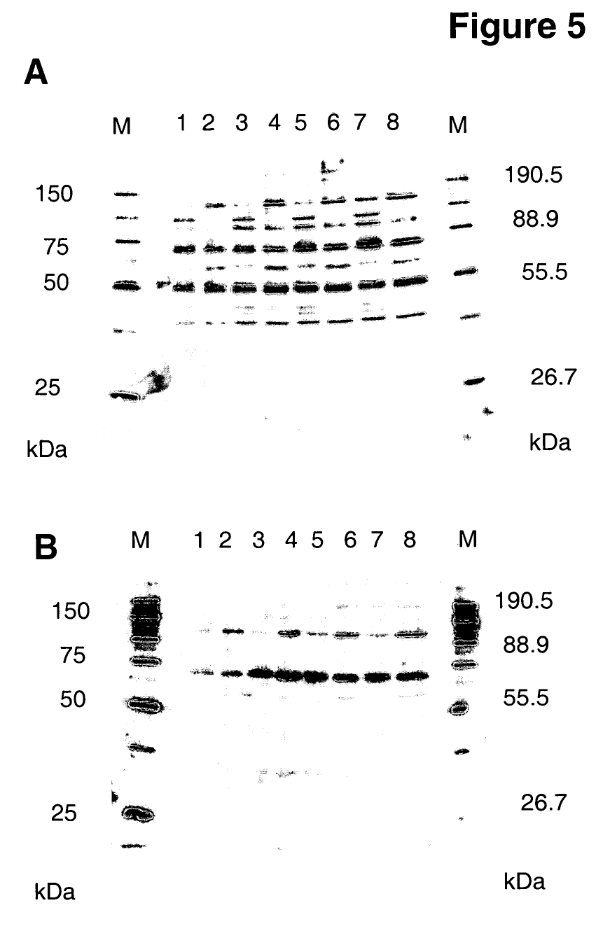

Using a “sandwich” method with dual-monoclonal assay as practiced for PSA analysis (McCormack RT, et al, “Molecular forms of prostate-specific antigen and the human kallikrein gene family: a new era”, Urology 45(5):729-44, 1995; Karazanashvili G, Abrahamsson P A, “Prostate specific antigen and human glandular kallikrein 2 in early detection of prostate cancer”, J. Urology 169(2): 445-457, 2003), a 49 kDa glycoprotein, reactive with RM2 antigen, was found as the major glycoprotein released from tumor cells, as evidenced by Western blot analysis. In addition, minor glycoprotein bands (88 kDa, 98 kDa, 130 kDa) were detected in various prostate cancer cell lines by Western blot analysis with RM2 (see FIG. 5A and its legend). These RM2-reactive glycoproteins were found in both androgen-dependent LNCap cells and androgen-independent PC3 cells. Combinations of RM2 and other monoclonal antibodies directed to non-RM2 epitopes expressed in these prostate cancer cell lines will be useful to set up efficient sandwich methods with a dual-monoclonal antibody assay.

Based on remarkable advances in electrospray ionization and matrix-assisted laser desorption/ionization mass spectrometry, these methods have been applied for analysis of tumor-associated glycoproteins in sera of patients with specific cancers, e.g., Johnson PJ, et al, “Structures of disease-specific serum alpha-fetoprotein isoforms”, Br. J. Cancer 83(10): 1330-1337, 2000; Poon TC, et al, “Comprehensive proteomic profiling . . . of hepatocellular carcinoma and its subtypes”, Clin. Chem. 49(5): 752-760, 2003. Along this line, SELDI-TOF-MS (surface-enhanced laser desorption/ionization-time of flight-mass spectrometry) is useful for characterization of glycoprotein antigens (Merchant M, Weinberger SR, “Recent advancements in surface-enhanced laser desorption/ionization-time of flight-mass spectrometry”, Electrophoresis 21: 1164-1167, 2000). Practically, RM2 glycoprotein in patient sera could be trapped with antibodies affixed on gel, followed by elution of adsorbed antigen, and ESI-MS, MALDI-MS, or SELDI-TOF-MS analysis.

Surface plasmon resonance spectroscopy is highly sensitive and capable of detecting weak interactions (Matsuura K, et al, “A quantitative estimation of carbohydrate-carbohydrate interaction . . . by surface plasmon resonance”, J. Am. Chem. Soc. 122(30): 7406-7407, 2000; Hernaiz MJ, et al, “A model system mimicking glycosphingolipid clusters to quantify carbohydrate self-interactions by surface plasmon resonance”, Angew. Chem. Intl. Ed. 41(9): 1554-1557, 2002). This is a promising approach for determination of antigen in patient serum by binding to antibody affixed on surface plasmon layer. E.g., a Fab derivative of RM2 antibody affixed on gold film (“self-assembled monolayer”; SAM) is used to detect antigen present in patient serum.

In preferred methods, the specimen is contacted with a moiety that specifically binds to RM2 antigen, and then the presence of the antigen is detected by detecting specific binding of the moiety to the RM2 antigen. Examples of moieties that specifically react with the RM2 antigen are antibodies that specifically bind to the RM2 antigen.

Within the context of the present invention, antibodies are understood to include polyclonal antibodies and monoclonal antibodies, single chain antibodies, antibody fragments (e.g., Fv, Fab, and F(ab′)₂), chimeric antibodies, resurfaced antibodies and humanized antibodies.

Polyclonal antibodies against the RM2 antigen may be readily generated by one of ordinary skill in the art from a variety of warm-blooded animals such as horses, cows, various fowl, rabbits, mice, hamsters, or rats. For example, a mammal, (e.g., a mouse, hamster, or rabbit) can be immunized with an immunogenic form of the RM2 antigen which elicits an antibody response in the mammal. The progress of immunization can be monitored by detection of antibody titers in plasma or serum. Following immunization, antisera can be obtained and polyclonal antibodies isolated from the sera.

Monoclonal antibodies are preferably used in the method of the invention. Monoclonal antibodies that specifically bind to RM2 antigen may be readily generated using conventional techniques. For example, monoclonal antibodies may be produced by the hybridoma technique originally developed by Kohler and Milstein 1975 (Nature 256, 495-497); see also U.S. Pat. No. RE 32,011, U.S. Pat. Nos. 4,902,614, 4,543,439, and 4,411,993 which are incorporated herein by reference; see also Monoclonal Antibodies, Hybridomas: A New Dimension in Biological Analyses, Plenum Press, Kennett, McKeam, and Bechtol (eds.), 1980, and Antibodies: A Laboratory Manual, Harlow and Lane (eds.), Cold Spring Harbor Laboratory Press, 1988). Other techniques may also be utilized to construct monoclonal antibodies (for example, see William D. Huse et al., 1989, “Generation of a Large Combinational Library of the Immunoglobulin Repertoire in Phage Lambda,” Science 246:1275-1281, L. Sastry et al., 1989 “Cloning of the Immunological Repertoire in Escherichia coli for Generation of Monoclonal Catalytic Antibodies: Construction of a Heavy Chain Variable Region-Specific cDNA Library,” Proc Natl. Acad. Sci. USA 86:5728-5732; Kozbor et al., 1983 Immunol. Today 4, 72 re the human B-cell hybridoma technique; Cole et al. 1985 Monoclonal Antibodies in Cancer Therapy, Allen R. Bliss, Inc., pages 77-96 re the EBV-hybridoma technique to produce human monoclonal antibodies; and see also Michelle Alting-Mees et al., 1990 “Monoclonal Antibody Expression Libraries: A Rapid Alternative to Hybridomas,” Strategies in Molecular Biology 3:1-9). Hybridoma cells can be screened immunochemically for production of antibodies specifically reactive with the RM2 antigen, and monoclonal antibodies can be isolated.

The term “antibody” as used herein is intended to include antibody fragments which are specifically reactive with RM2 antigen. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

Single chain antibodies may be produced by joining variable heavy and variable light chains with a linker (see, e.g., Huston et al.1988 Proc. Natl. Acad. Sci. U.S.A., 85, 5879-5883 and Bird et al. 1988 Science, 242, 423-426, which are incorporated herein by reference).

The invention also contemplates chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of differentiated cells or tumor cells. See, for example, Morrison et al., 1985; Proc. Natl. Acad. Sci. U.S.A. 81, 6851; Takeda et al., 1985, Nature 314, 452; Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B.

The invention further contemplates the use of resurfaced monoclonal antibodies. Methods of resurfacing antibodies are described in the literature for example, see U.S. Pat. No. 5,639,641, expressly incorporated herein by reference.

Humanized antibodies can also be used in the present method. Methods of humanizing antibodies are well known in the art and are described in the literature, for example, Padlan, E. et al. 1991 Molecular Immunology, vol. 28, pp. 489-498, U.S. Patent Publication 2002.0034765 A1, and U.S. Patent Publication 2004/0058414 A1.

Thus, suitable antibodies for use in the method of the present invention include polyclonal antibodies, single chain polyclonal antibodies, polyclonal antibody fragments, monoclonal antibodies, single chain monoclonal antibodies, monoclonal antibody fragments, chimeric antibodies, single chain chimeric antibodies, chimeric antibody fragments, resurfaced antibodies, resurfaced single chain antibodies, resurfaced antibody fragments, humanized antibodies, humanized single chain antibodies, and humanized antibody fragments.

Especially preferred for use in the present invention is RM2 mAb and fragments thereof. Monoclonal antibody RM2 and methods of making it are described in Saito, S., Levery, S. B., Salyan, M. E. K., Goldberg, R. I., and Hakomori, S. 1994 J. Biol. Chem. 269, 5644-5652, which are incorporated herein by reference.

Methods for detecting specific binding of antibody to the RM2 antigen are well known in the art and include immunohistology; sodium dodecyl sulfate polyacrylamide gel electrophoresis (SDS-PAGE) followed by Western Blot analysis; labeled secondary antibody directed to primary antibody that binds to said antigen; surface plasma resonance (SPR) spectroscopy; and molecular force microscopy.

E. Kit for diagnosing prostate cancer: The present invention also provides a kit for diagnosing prostate cancer, comprising:
(a) At least one moiety that specifically binds to RM2 antigen, having the epitope structure shown below, from a specimen obtained from a patient suspected of having prostate cancer: 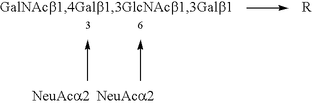
   wherein R represents a carrier,
(b) Instructions for diagnosing prostate cancer using said kit, and
(c) Optionally, a means for detecting the presence of said antigen by specific binding of said moiety to said antigen.

Suitable carriers are those described above.

Suitable moieties that specifically bind to RM2 antigen can be any of those described for use in the method of diagnosis.

Suitable means for detecting are those described for the method of diagnosis.

Instructions include the types of specimens suitable for diagnostic assay, such as those described above for the method of diagnosis.

F. Composition of matter: The present invention also provides an isolated or purified prostate tissue sample comprising RM2 antigen. The tissue sample is isolated and/or purified by methods known in the art. Isolation methods for glycosphingolipid and glycoprotein antigens are summarized in Hakomori S & Kannagi R, “Carbohydrate antigens in higher animals”, in: Handbook of Experimental Immunology; Vol. 1: Immunochemistry (Weir DM, Herzenberg L A, Blackwell C, Herzenberg L A, eds.), 4th ed., Blackwell Scientific Publications (Oxford; Boston), chap. 9 (pp. 9.1-9.39). As pointed out above, the antigen is a glycoprotein (Mr ˜50 kDa). This is significant, because in many cases, glycosphingolipid antigens are not released at high level, as compared with glycoprotein antigens.

The tissue sample, whether purified, isolated or not, can be used to make monoclonal antibodies that specifically bind to RM2 antigen by methods well known in the art. See for example, Saito, S., Levery, S. B., Salyan, M. E. K., Goldberg, R. I., and Hakomori, S. 1994 J. Biol. Chem. 269, 5644-5652, which is incorporated herein by reference.

All publications and patent applications are herein incorporated by reference to the same extent as if each individual publication or patent application was specifically and individually indicated to be incorporated by reference. Although the present invention has been described in some detail by way of illustration and example for purposes of clarity and understanding, it will be apparent that certain changes and modifications may be practiced within the scope of the appended claims.

1. A method for diagnosing prostate cancer, comprising detecting the presence of or elevated levels of RM2 antigen, having the epitope structure shown below, in a specimen from a patient suspected of having prostate cancer: 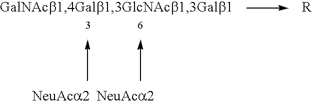
wherein R represents a carrier.

2. The method of claim 1, wherein said method further comprises contacting said specimen with at least one antibody that specifically binds to said RM2 antigen, and detecting the presence of said antigen by specific binding of antibody to antigen

3. The method of claim 2, wherein said at least one antibody is selected from the group consisting of a polyclonal antibody, a single chain polyclonal antibody, a polyclonal antibody fragment, a monoclonal antibody, a single chain monoclonal antibody, a monoclonal antibody fragment, a chimeric antibody, a single chain chimeric antibody, a chimeric antibody fragment, a resurfaced antibody, a resurfaced single chain antibody, a resurfaced antibody fragment, a humanized antibody, a humanized single chain antibody, and a humanized antibody fragment.

4. The method of claim 2, wherein said at least one antibody is a monoclonal antibody.

5. The method of claim 2, wherein said at least one antibody is directed to the epitope recognized by RM2 monoclonal antibody.

6. The method of any one of claims 1 or 2, wherein said specimen is a prostate biopsy sample.

7. The method of any one of claims 1 or 2, wherein said specimen is a specimen from a total prostatectomy.

8. The method of any one of claims 1 or 2, wherein said specimen is a serum sample.

9. The method of claim 2, wherein the presence of said antigen is detected by immunohistology; sodium dodecyl sulfate polyacrylamide gel electrophoresis (SDS-PAGE) followed by Western Blot analysis; labeled secondary antibody directed to primary antibody that binds to said antigen; surface plasma resonance (SPR) spectroscopy; or molecular force microscopy.

10. The method of claim 2, wherein said specimen is a prostate biopsy sample and the presence of said antigen is detected via immunohistology, labeled secondary antibody directed to primary antibody that binds to said antigen, surface plasma resonance (SPR) spectroscopy, or molecular force microscopy.

11. The method of claim 2, wherein said specimen is a specimen from a total prostatectomy and the presence of said antigen is detected via immunohistology, labeled secondary antibody directed to primary antibody that binds to said antigen, surface plasma resonance (SPR) spectroscopy, or molecular force microscopy.

12. The method of claim 2, wherein said specimen is a serum sample and the presence of said antigen is detected via SDS-PAGE followed by Western blot analysis.

13. The method of claim 10, wherein said at least one antibody is directed to the epitope recognized by RM2 monoclonal antibody.

14. The method of claim 11, wherein said at least one antibody is directed to the epitope recognized by RM2 monoclonal antibody.

15. The method of claim 12, wherein said at least one antibody is directed to the epitope recognized by RM2 monoclonal antibody.

16. A kit for diagnosing prostate cancer, comprising:
(a) At least one moiety that specifically binds to RM2 antigen, having the epitope structure shown below, from a specimen obtained from a patient suspected of having prostate cancer: 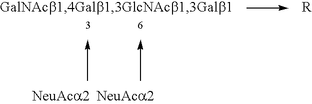
wherein R represents a carrier,
(b) Instructions for diagnosing prostate cancer using said kit, and
(c) Optionally, a means for detecting the presence of said antigen by specific binding of said moiety to said antigen.

17. The kit of claim 16, wherein the moiety that specifically binds to said RM2 antigen is an antibody.

18. The kit of claim 16, wherein said antibody is selected from the group consisting of a polyclonal antibody, a single chain polyclonal antibody, a polyclonal antibody fragment, a monoclonal antibody, a single chain monoclonal antibody, a monoclonal antibody fragment, a chimeric antibody, a single chain chimeric antibody, a chimeric antibody fragment, a resurfaced antibody, a resurfaced single chain antibody, a resurfaced antibody fragment, a humanized antibody, a humanized single chain antibody, and a humanized antibody fragment.

19. The kit of claim 16, wherein said moiety that specifically binds to said RM2 antigen is an antibody.

20. The kit of claim 16, wherein said moiety that specifically binds to said RM2 antigen is a monoclonal antibody.

21. The kit of claim 16, wherein said moiety that specifically binds to said RM2 antibody is directed to the epitope recognized by RM2 monoclonal antibody.

22. The kit of any one of claims 16 or 17, wherein said specimen is a prostate biopsy sample.

23. The kit of any one of claims 16 or 17, wherein said specimen is a specimen from a total prostatectomy.

24. The kit of any one of claims 16 or 17, wherein said specimen is a serum sample.

25. The kit of claim 16, wherein the presence of said antigen is detected via immunohistology; sodium dodecyl sulfate polyacrylamide gel electrophoresis (SDS-PAGE) followed by Western Blot analysis; labeled secondary antibody directed to primary antibody that binds to said antigen; surface plasma resonance (SPR) spectroscopy; or molecular force microscopy.

26. The kit of claim 17, wherein said specimen is a prostate biopsy sample and the presence of said antigen is detected by immunohistology; labeled secondary antibody directed to primary antibody that binds to said antigen; surface plasma resonance (SPR) spectroscopy; or molecular force microscopy.

27. The kit of claim 17, wherein said specimen is a specimen from a total prostatectomy and the presence of said antigen is detected by immunohistology; labeled secondary antibody directed to primary antibody that binds to said antigen; surface plasma resonance (SPR) spectroscopy; or molecular force microscopy.

28. The kit of claim 17, wherein said specimen is a serum sample and the presence of said antigen is detected via SDS-PAGE followed by Western blot analysis.

29. The kit of claim 17, wherein said specimen is a sample from a body secretion and the presence of said antigen is detected via SDS-PAGE followed by Western blot analysis.

30. The kit of claim 26, wherein said at least one antibody is directed to the epitope recognized by RM2 monoclonal antibody.

31. The kit of claim 27, wherein said at least one antibody is directed to the epitope recognized by RM2 monoclonal antibody.

32. The kit of claim 28, wherein said at least one antibody is directed to the epitope recognized by is RM2 monoclonal antibody.

33. An isolated-prostate tissue sample comprising RM2 antigen.
